(19) United States
(12) Patent Application Publication
(10) Pub. No.: US 2022/0047803 A1
(43) Pub. Date: Feb. 17, 2022

(54) OSMOTIC ACTUATOR FOR A WEARABLE INJECTION DEVICE AND A WEARABLE INJECTION DEVICE COMPRISING SUCH AN OSMOTIC ACTUATOR

(71) Applicants: Subcuject ApS, Hellebæk (DK); Porifera Inc., San Leandro, CA (US)

(72) Inventors: Ravindra REVANUR, Fremont, CA (US); Claus Schmidt MØLLER, Fredensborg (DK)

(21) Appl. No.: 17/276,073

(22) PCT Filed: Sep. 5, 2019
(86) PCT No.: PCT/DK2019/050263

§ 371 Date: Mar. 12, 2021

Related U.S. Application Data
(60) Provisional application No. 62/730,805, filed on Sep. 13, 2018.

(51) Int. Cl. A61M 5/142; A61M 5/145

(52) U.S. Cl. CPC A61M 5/14248; A61M 2205/584; A61M 2005/14513; A61M 5/145

(57) ABSTRACT

An osmotic actuator (110) for a wearable injection device (100) is disclosed, which osmotic actuator comprises a pressure chamber (150) having one or more outlets (152) and containing a draw solution containing dissolved inorganic salt, one or more osmotic membranes (130), and a cavity (140) containing water, and wherein the one or more osmotic membranes (130) form at least a part of one or more internal surfaces of the pressure chamber (150), wherein the water is in contact with at least a part of one or more external surfaces of the one or more osmotic membranes (130), and wherein the dissolved inorganic salt in the draw solution comprises one or more of the following: CaBr₂, CaCl₂, ZnBr₂, ZnCl₂, ZnI₂ or LiBr. Furthermore, a wearable injection device (100) comprising such an osmotic actuator (110) and the use of such inorganic salts in a draw solution are disclosed.

### Field of invention

The present invention relates to an osmotic actuator for a wearable injection device and to a wearable injection device comprising such an osmotic actuator. In particular, it relates to an osmotic actuator, which is capable of providing a high and stable flow rate during the use of the wearable injection device.

### BACKGROUND OF THE INVENTION

Using an osmotic actuator based on forward osmosis as the drive unit in an injection device is very attractive in situations, in which the drug must be injected slowly into the patient, e.g. when a large volume of drug must be injected. An osmotic actuator is capable of providing a high pressure, but at the same time the time for building up the pressure can be controlled by the type and size of the osmotic membrane and by the concentration and type of the osmotic draw solution. The pressure and the increased volume in the actuator due to feed water passing through the osmotic membrane and into the actuator can, for instance, be used for moving a plunger in a cartridge or to squeeze drug out of a flexible reservoir.

WO 2017/129191 A1 describes several embodiments of a wearable injection device driven by an osmotic drive unit. One internal side of a pressure chamber containing a draw solution is formed by an osmotic membrane, which is in contact with a feed water reservoir on the outside. When feed water is drawn into the chamber through the membrane due to the osmotic process, a pressure builds up and the excess fluid is pressed out of the osmotic drive unit through an outlet and arranged to move the plunger in a cartridge. However, a disadvantage of such wearable devices known in the art is that it is difficult to get the dimensions as small as desired to ease the handling of the device and the convenience in use. Furthermore, it is difficult to provide a sufficient flow rate for a conveniently sized device.

### BRIEF DESCRIPTION OF THE INVENTION

It is an objective of the invention to provide an osmotic actuator for a wearable injection device, which overcomes the above-mentioned disadvantages related to osmotic actuators known in the prior art.

The present invention relates to an osmotic actuator for a wearable injection device, which osmotic actuator comprises a pressure chamber having one or more outlets and containing a draw solution containing dissolved inorganic salt, one or more osmotic membranes, and a cavity containing water, and wherein the one or more osmotic membranes form at least a part of one or more internal surfaces of the pressure chamber, wherein the water is in contact with at least a part of one or more external surfaces of the one or more osmotic membranes, and wherein the dissolved inorganic salt in the draw solution comprises one or more of the following: CaBr₂, CaCl₂, ZnBr₂, ZnCl₂, ZnI₂ or LiBr.

By using such inorganic salts in the draw solution, small dimensions of the actuator and, thereby, of the wearable injection device are achievable and it is easier to obtain a high and constant flow rate.

In an embodiment of the invention, the draw solution further contains other types of salts or other osmotic agents.

Such other contents of the draw solution could, for instance, be NaCl or a polymer. Even a saturated solution of CaBr₂ and or, e.g. ZnBr₂ can dissolve smaller amounts of other types of salt or other osmotic active ingredients, whereby the osmotic potential can be further increased.

In an embodiment of the invention, the draw solution further contains an alcohol.

By mixing the draw solution with an alcohol, the density of the draw solution can be reduced, which might be an advantage in some wearable injection device designs and, at the same time, alcohol will act as a wetting agent increasing the mixing speed with the incoming water.

In another aspect of the invention, it relates to a wearable injection device comprising an osmotic actuator as described above.

In an embodiment of the invention, the draw solution is contained in a tight cavity inside the pressure chamber and surrounded by water, and wherein the draw solution is arranged to be pressed out of the cavity and mixed with the surrounding water as part of an activation sequence for the wearable injection device.

In this way, an equilibrium state is provided until the wearable injection device is activated, and the wearable injection device can thereby be stored for a long time before being used.

In an embodiment of the invention, crystal salt is being dissolved in the pressure chamber as part of an activation sequence for the wearable injection device.

By releasing crystal salt in the surrounding water in the pressure chamber as part of the activation sequence, a more simple activation mechanism for the wearable injection device is achievable.

In an embodiment of the invention, the draw solution at least in a part of the osmotic actuator is supersaturated when the wearable injection device has been activated.

By supersaturating the draw solution at activation of the wearable injection device, the dilution of the draw solution during the use of the wearable injection device can be more or less equalized, which results in a more stable flow rate.

In an embodiment of the invention, a colour agent is mixed with the draw solution and colouring the draw solution when the wearable injection device is activated.

This can help the user to determine the progress of the injecting as the coloured draw solution moves into a drug-filled container advancing the plunger. If the wearable injection device is arranged to open the pressure chamber to the cavity containing the feed water when the injection is fulfilled, then the wearable injection device can be arranged to show the colour in a window, thus indicating to the user that the injection has been completed.

In an embodiment of the invention, fluid leaving the pressure chamber through the one or more outlets is arranged to press a drug out of a drug-filled container of the wearable injection device by means of a movable plunger.

In this way, a rod for pushing the plunger is avoided and the wearable injection device can be made substantially smaller.

In an embodiment of the invention, fluid leaving the pressure chamber through the one or more outlets is arranged to press a drug out of a flexible reservoir of the wearable injection device.

With such a configuration, a very compact wearable injection device based on a flexible drug reservoir can be provided.

In yet another embodiment of the invention, it relates to a use of one or more of the following inorganic salts: CaBr₂, CaCl₂, ZnBr₂, ZnCl₂, ZnI₂ or LiBr in a draw solution in an osmotic actuator.

In an embodiment of the invention, the osmotic actuator is arranged to be used in a wearable injection device.

In an embodiment of the invention, the draw solution further contains other types of salts or other osmotic agents.

In an embodiment of the invention, the draw solution further contains an alcohol.

### DRAWINGS

In the following, a few exemplary embodiments of the invention are described in further detail with reference to the drawings, of which

FIG. 1 is a perspective view of a wearable injection device according to an embodiment of the invention,

FIG. 2 is a perspective view of an osmotic actuator according to an embodiment of the invention,

FIG. 3 shows a curve illustrating the loss of osmotic potential due to concentration polarisation, and

FIG. 4 is a table showing the solubility limits of NaCl, CaBr₂ and ZnBr₂ at different temperatures.

### DETAILED DESCRIPTION

Only the parts necessary to understand the function of the osmotic actuator 110 are included in the description.

The terms “up”, “down”, “upper”, “lower”, “upward” and “downward” refer to the drawings and not necessarily to a situation of use.

The term “wearable injection device” 100 refers to a patient-administrated medical injection device 100 for attachment to the body and for subcutaneous injection of a medicament. A wearable injection device 100 injects at a lower speed than e.g. an auto-injector and is often used when large amounts of drug must be injected. Wearable injection devices 100 are often for one-time use and are removed and disposed with after use.

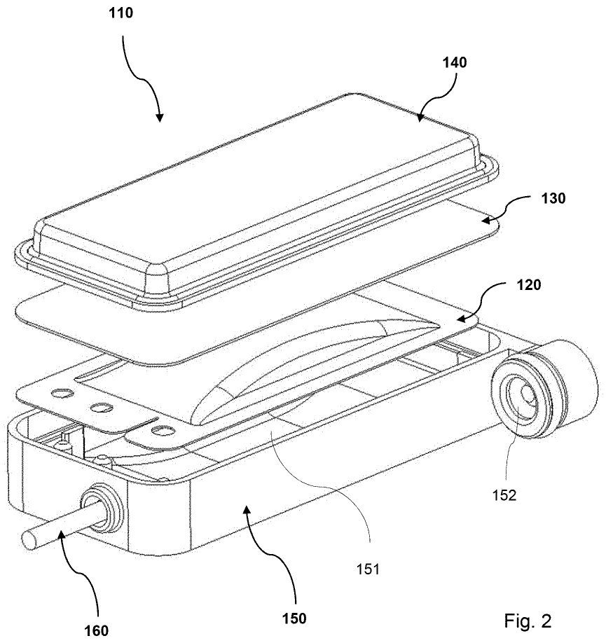

The term “osmotic actuator” 110 can refer to an osmotic actuator 110 with a flat sheet osmotic membrane 130 as shown in FIG. 2, but also to an osmotic actuator 110 with two or more flat sheet osmotic membranes 130. The osmotic actuator 110 comprises a solvent supply, e.g. in the form of a flexible reservoir 140 with feed water, and a rigid pressure chamber 150 with one or more outlets 152 and containing a draw solution. During use, the feed water from the flexible reservoir 140 passes through the osmotic membrane 130 into the pressure chamber 150, whereby the pressure in the pressure chamber 150 increases.

The term “FO” means Forward Osmosis as opposed to the term RO, which means Reverse Osmosis. The forward osmosis process runs without any additional energy being applied, whereas the reverse osmosis process requires the application of a high pressure.

The term “flat sheet membrane” 130 or simply “membrane” 130 refers to a semipermeable FO membrane 130 adapted to initiate an osmotic pressure in an osmotic actuator 110 by means of FO. The flat sheet membrane 130 may be bent or shaped and it may also be in the form of tubular or hollow fibre membranes 130 where this is considered advantageously.

The term “feed water” refers to water with a lower salinity or a lower osmotic potential than the draw solution. The feed water is preferably in the form of demineralized water, but the term might also refer to other kinds of fluids. It might also simply be referred to as “water”. The feed water is contained in a flexible or collapsible reservoir 140 also referred to as “a cavity 140 containing water”.

The term “draw solution” refers to a solution containing an osmotic agent and with a higher salinity or osmotic potential than the feed water. At activation of the wearable injection device 100, the draw solution can either be released from a reservoir 120 or be made by dissolving an osmotic agent, e.g. crystal salt as powder or stamped to form a tablet.

The term “DECP” or “Dilutive External Concentration Polarization” refers to the phenomenon that water from the water reservoir 140, which passes through the semipermeable FO membrane 130 and into the pressure chamber 150 with the draw solution accumulates near the membrane 130 surface, whereby the osmotic potential falls.

The term “CICP” or “Concentrative Internal Concentration Polarization” refers to the phenomenon that osmotic agents from the draw solution passes through the semipermeable FO membrane 130 and into the feed water reservoir 140 and accumulates inside the porous support layer, whereby the osmotic potential falls.

The term “PRO” or “PRO mode” refers to an orientation of the osmotic forward osmosis membrane 130, where the active layer of the membrane 10 is facing the draw solution and the porous support layer is facing the feed water.

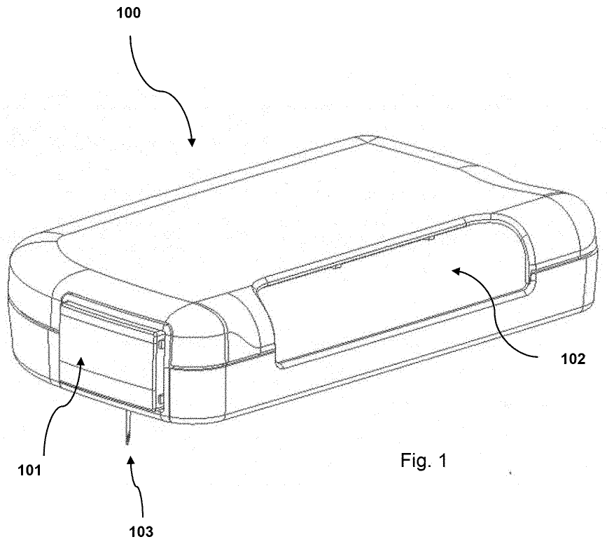

FIG. 1 shows a wearable injection device 100 comprising an osmotic actuator 110 (shown in FIG. 2). The wearable injection device 100 is in its activated state, in which the push-button 101 for activating the wearable injection device 100 is pushed, and the needle 103 is in the extended position, in which it is inserted in the subcutaneous tissue of a patient. The drug-filled container 102 can be seen through an opening in the wearable injection device 100.

The functional sequences of a wearable injection device 100 as the one shown in FIG. 1 may, for instance, be as follows:
The user peels off the protection paper of the adhesive on the user-interfacing side of the wearable injection device 100.
The user attaches the wearable injection device 100 to the body, normally in the abdominal region.
The user pushes the push-button 101, whereby the hypodermic needle 103 in inserted into the subcutaneous skin of the user and a flow path is created between the drug-filled container 102 and the user.
During and immediately after the push of the push-button 101, the salt or salt solution is released within the osmotic actuator 110 (see FIG. 2) to form the draw solution in the pressure chamber 150. Feed water is drawn through an osmotic FO membrane 130 (see FIG. 2) and the excess fluid consisting of a mix of feed water and draw solution is lead to the drug-filled container 102.
The plunger (not shown) in the drug-filled container 102 is moved by the excess fluid pressing onto the external surface of the plunger, and the drug is pushed out through the hypodermic needle 103.
When the injection is fulfilled, the needle 103 may be retracted automatically and a signal that indicates that the injection is fulfilled may be given to the user.
The user removes the wearable injection device 100 and disposes of it.

FIG. 2 shows more details of an osmotic actuator 110 according to an embodiment of the invention. As can be seen, the osmotic actuator 110 comprises a pressure chamber 150 with an outlet 152, which is adapted to be connected with a drug-filled container 102 (shown in FIG. 1). The pressure chamber has as a cavity or compartment 151 containing a pouch 120 with the draw solution and with water surrounding the pouch 120. An osmotic membrane 130 and a collapsible feed water reservoir 140 are arranged on the upper part of the pressure chamber 150, so that the membrane 130 constitutes the barrier between the pressure chamber 150 and the feed water reservoir 140. The pouch 120 with the draw solution is positioned and fixed within the actuator 110. A cutting device 160 is cutting the pouch 120 with the draw solution open when the wearable injection device 100 is activated by a push on the push-button 101 (see FIG. 1), and the pouch 120 is emptied, e.g. by means of elastomeric properties of the pouch 120 or by means of a spring (not shown). The draw solution will hereafter mix with the surrounding water in the pressure chamber 150.

In other embodiments of the invention, the release of the draw solution in the pressure chamber 150 can be carried out in other ways, for instance by means of either a dry or a dissolved osmotic agent that is mixed with surrounding water at activation of the wearable injection device 100.

One big difference between the use of osmotic membranes 130 in the above-described wearable injection device 100 and the use in, e.g., water treatment is that, in the wearable injection device 100, the feed water and the draw solution are generally static during the use of the wearable injection device 100, and there is generally no fluid movement along the membrane 130. This has the consequence that the osmotic potential and thereby the driving force falls during the use of the wearable injection device 100 due to Dilutive External Concentration Polarisation (DECP) and Concentrative Internal Concentration Polarization (CICP). The DECP and CICP must be subtracted from the apparent osmotic potential between the draw solution and the feed water and the resulting available osmotic potential is thereby lower.

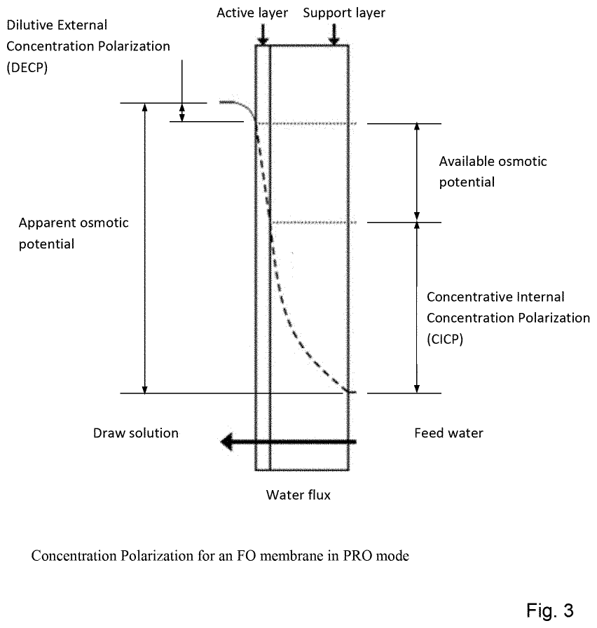

In FIG. 3, the effect of DECP and CICP is illustrated for a membrane 130 in PRO mode, which is the mode that provides the highest available osmotic potential when the draw solution and feed water are generally static. As indicated in FIG. 3, DECP and CICP can have a significant and increasing effect during the use of the wearable injection device 100 until an equilibrium occurs. This may potentially have the consequence that the driving force becomes insufficient at the final part of the injection so that the injection stops or that the dose rate slows down too much, due to reduced water transfer through the membrane 130.

By using a draw solution based on calcium bromide (CaBr₂) dissolved in water, an initial osmotic potential that is much higher than by using NaCl or MgCl₂ or other commonly used osmotic agents is achieved. As a result thereof, the water transfer through the membrane 130 is maintained at a sufficiently high level throughout the entire injection period. The draw solution may be composed by CaBr₂dissolved in water or by a combination/composition with zinc bromide (ZnBr₂), zinc chloride (ZnCl₂), zinc iodide (ZnI₂), lithium bromide (LiBr) or calcium chloride (CaCl₂) or with additional common salts or other osmotic active ingredients. A draw solution made by one or more of ZnBr₂, ZnCl₂, ZnI₂, LiBr or CaCl₂ alone and without the presence of CaBr₂ is also within the scope of the invention.

In order to achieve a sufficiently high water-transfer through an FO membrane 130, there are three important factors to be considered:
1) A high number of dissolved particles in the draw solution
2) A constant transportation of particles to the membrane (to avoid DECP)
3) A high membrane rejection of the particles

### Re 1

For draw solutions based on dissolved salts, such as CaBr₂, the “particles” in the draw solution are in the form of ions and, therefore, the solubility of the salt is a very important factor in defining the total number of “particles” that the draw solution can contain. CaBr₂ and the other salts mentioned above are highly soluble in water and can therefore provide a considerable number of ions per millilitre. Compared to NaCl, CaBr₂ has a 4-7 times higher solubility and ZnBr₂ has a 10-20 higher solubility in water.

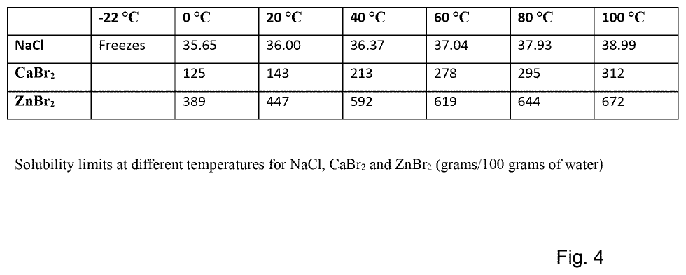

In FIG. 4, the approximate solubility limits at different temperatures of NaCl, CaBr₂ and ZnBr₂ in water can be seen, the unit being gram of salt/100 gram of water.

A salt solution, which is saturated at 0° C., has a crystallization temperature at 0° C. The crystallization temperature is defined as the temperature, at which the salt crystals begin to fall out of the solution given sufficient time and proper nucleating conditions. Once formed, masses of salt crystals are difficult to remove and can block the osmotic actuator 110. Therefore, it is desirable to have a crystallization temperature below 0° C. Below, the solubility of NaCl, CaBr₂ and ZnBr₂ are shown as weight percentage (wt %: weight of salt divided by weight of solution) at 0° C.:

| | NaCl | CaBr₂ | ZnBr₂ |
|---|---|---|---|
| Wt % at 0° C. | 25 | 52 | 76 |
| (saturated solution) | | | |

It is clear that the wt % for a CaBr₂ solution and especially for a ZnBr₂ solution is considerably higher than for an NaCl solution. In many cases, a composition of two or more salts may be used to increase the total solubility and, interestingly, a blend of 54-57 wt % of ZnBr2 and 21-23 wt % of CaBr₂ with a resulting weight percentage of 75 wt % has a crystallization temperature at −12° C., whereby it can be stored a longer time and at a much lower temperature without the risk of crystals growing in the draw solution during storage.

Below, the water transfer in a test cell with a single flat FO membrane 130 with an effective area of app. 1000 mm² is shown for NaCl, CaBr₂ and a ZnBr₂—CaBr₂ (5:2) blend for app. 90% saturated solutions:

| | Water transfer |
|---|---|
| Salt: | (gram/5 min) at 20° C. |
| NaCl | 4 |
| CaBr₂ | 9.5 |
| ZnBr₂—CaBr₂ | 11.2 |

### Re 2

As the draw solution and the feed water are generally static in the wearable injection device 100 during use, all potential turbulence and movements of the fluids will arise from the feed water coming through the membrane 130 and from the user moving while using the wearable injection device 100. This means that the bulk diffusion rate of the salt in the solution is important to ensure that particles moves towards the membrane 130. The diffusion rate of a salt is related to the size and weight of the ions of the salt, and as the weights of the ions of CaBr₂ and ZnBr₂ and the other mentioned salts are high compared to the ions of NaCl, the bulk diffusion rate is relatively low for these salts.

However, also density is important in an osmotic actuator 110 with stationary fluids. When the draw solution is much heavier than the incoming feed water (and because the mixing of the incoming water and the draw solution does not happen instantly), the draw solution tends to fall through the incoming water and down to the membrane 130 when the draw solution is oriented above the membrane 130 and to a certain degree when the membrane 130 is oriented vertically. This minimizes the DECP significantly and increases the available osmotic potential as, thereby, ions are led to the membrane 130. In PRO mode, the CICP primarily happens in the support layer, as shown in FIG. 3, and the support layer should therefore be as thin and open as possible. A higher density of the CaBr₂ will also help to remove the ions from the support layer of the membrane 130 as they will tend to be moved away by gravity with the above described orientations of the membrane 130. If an amount of air is present within the osmotic actuator 110, a high density of the draw solution can likewise help pushing away air pockets from the membrane 130 surface.

Below, the densities of NaCl, CaBr₂ and a ZnBr₂—CaBr₂ blend at 20° C. are listed:

| | Density of a saturated |
|---|---|
| | solution (gram/mL) |
| NaCl | 1.22 |
| CaBr₂ | 1.78 |
| ZnBr₂—CaBr₂ | 2.3 |

### Re 3

Typically, the bigger size of the ions, the higher rejection of the ions (particles) by the membrane 130. With a low rejection, the transfer of ions from the pressure chamber 150 to the feed water reservoir will be high, which will reduce the effective driving force due to CICP. As the molecule size of the claimed salts is rather high, the rejection of the ions is very good. For example, the Reverse Salt Flux (RSF) for CaBr₂ is app. 0.18 gram/L compared to app. 0.3 gram/L for NaCl, measured on a commercially available FO membrane 130 from Porifera Inc. under standard FO test conditions.

When the water enters the pressure chamber 150 through the membrane 130, it will to some extend mix with the draw solution due to the bulk diffusion as described earlier and, therefore, the water passing through the outlet 152 will to some extend be a diluted draw solution. This dilution further amplifies the issue with the falling flux over the membrane 130. Due to the relatively high density of the CaBr₂ solution compared with the feed water, it is possible to trap the CaBr₂ solution inside the pressure chamber 150, e.g. by forming the pressure chamber 150 or part of the pressure chamber 150 as a spiral with the outlet 152 in the middle. In a vertical position of the membrane 130, the high density of the draw solution will then maintain the most saturated draw solution in the spiral formed channel. To have the same effect in a horizontal orientation of the membrane 130, a more complex design of the osmotic actuator 110 might be envisioned.

Another advantage of CaBr₂ and ZnBr₂, ZnCl₂, ZnI₂, LiBr and CaCl₂ over other osmotic agents is the relatively high solubility in ethanol and other alcohols, especially compared to other types of salt. In some examples, it may be an advantage to lower the density of the draw solution and bring it closer to the density of water, and this may be achieved, for instance, by adding ethanol with a density of 0.79 g/mL or other alcohols to the draw solution. A smaller amount of alcohol in the draw solution may have the additional advantage of improving wetting and thereby the diffusion, which will catalyse a faster mixing of the draw solution and the incoming feed water.

Further advantages related to using especially CaBr₂ salt in the draw solution are:
It is not hazardous and can easily be disposed with without damage to the environment.
It is thermally and chemically stable, nonflammable and not explosive.
No toxic fumes are created in case of fire.
Low thermal expansion in a broad temperature range helps ensuring a stable flow at different temperatures.
Even with a saturated solution, it is possible to dissolve smaller amounts of other types of salt and the osmotic potential can thereby be further increased.
It is relatively cheap and easily accessible. CaBr₂ is mainly used in dense aqueous solutions for drilling fluids, but it is also used in neurosis medication, freezing mixtures, food preservatives, photography and fire retardants.
CaBr₂ releases a relatively high amount of heat when the anhydrous form is dissolved in water, and this can be used to heat up the osmotic actuator 110 resulting in a higher water transfer through the membrane 130.
Dissolved CaBr₂ is clear and does not smell.

An osmotic actuator 110 based on a CaBr₂ draw solution may also be used in other kinds of devices or pumps both inside and outside the pharmaceutical industry.

### LIST OF REFERENCE NUMBERS

100. wearable injection device
101. Push-button
102. Drug-filled container
103. Needle
110. Osmotic actuator
120. Cavity containing draw solution
130. Osmotic membrane
140. Cavity containing water
150. Pressure chamber
151. Compartment in pressure chamber
152. Outlet
160. Cutting device

1. An osmotic actuator for a wearable injection device, which osmotic actuator comprises
a pressure chamber having one or more outlets and containing
a draw solution containing dissolved inorganic salt,
one or more osmotic membranes, and
a cavity containing water, and
wherein the one or more osmotic membranes form at least a part of one or more internal surfaces of the pressure chamber,
wherein the water is in contact with at least a part of one or more external surfaces of the one or more osmotic membranes, and
wherein the dissolved inorganic salt in the draw solution comprises one or more of the following: CaBr₂, CaCl₂, ZnBr₂, ZnCl₂, ZnI₂ or LiBr.

2. The osmotic actuator according to claim 1, wherein the draw solution further contains other types of salts or other osmotic agents.

3. The osmotic actuator according to claim 1, wherein the draw solution further contains an alcohol.

4. A wearable injection device comprising an osmotic actuator according to claim 1.

5. The wearable injection device according to claim 4, wherein the draw solution is contained in a tight cavity inside the pressure chamber and surrounded by water, and wherein the draw solution is arranged to be pressed out of the cavity and mixed with the surrounding water as part of an activation sequence for the wearable injection device.

6. The wearable injection according to claim 4, wherein crystal salt is being dissolved in the pressure chamber as part of an activation sequence for the wearable injection device.

7. The wearable injection device according to claim 6, wherein the draw solution at least in a part of the osmotic actuator is supersaturated when the wearable injection device has been activated.

8. The wearable injection device according to claim 5, wherein a colour agent is mixed with the draw solution and colouring the draw solution when the wearable injection device is activated.

9. The wearable injection device according to claim 4, wherein fluid leaving the pressure chamber through the one or more outlets is arranged to press a drug out of a drug-filled container of the wearable injection device by means of a movable plunger.

10. The wearable injection device according to claim 4, wherein fluid leaving the pressure chamber through the one or more outlets is arranged to press a drug out of a flexible reservoir of the wearable injection device.

11. A draw solution in the osmotic actuator according to claim 1 comprising one or more of the following inorganic salts:
CaBr₂, CaCl₂, ZnBr₂, ZnCl₂, ZnI₂ or LiBr.

12. The draw solution according to claim 11, wherein the osmotic actuator is arranged to be used in a wearable injection device.

13. The draw solution according to claim 11 further comprising other types of salts or other osmotic agents.

14. The draw solution according to claim 11, further comprising an alcohol.

15. The wearable injection device according to claim 6, wherein a color agent is mixed with the draw solution, which colors the draw solution when the wearable injection device is activated.
